(19) United States
(12) Patent Application Publication
(10) Pub. No.: US 2026/0083440 A1
(43) Pub. Date: Mar. 26, 2026

(54) MULTI-PHASE SAMPLE COLLECTION DEVICE

(71) Applicant: IDEXX Laboratories, Inc., Westbrook, ME (US)

(72) Inventor: Justin Griffin, Scarborough, ME (US)

(73) Assignee: IDEXX Laboratories, Inc., Westbrook, ME (US)

(21) Appl. No.: 19/248,621

(22) Filed: Jun. 25, 2025

Related U.S. Application Data
(60) Provisional application No. 63/698,119, filed on Sep. 24, 2024.

(51) Int. Cl. A61B 10/00

(52) U.S. Cl. CPC A61B 10/0038

(57) ABSTRACT

A device for collecting a multi-phase sample. The device includes a solid sample capture device. The solid sample capture device includes a grip, an elongated member, and a solid sample collection structure. The device further includes a liquid sample capture device integrated into the solid sample capture device. The liquid sample capture device includes a suction component integrated into the grip, a tube integrated into the elongated member, and a tip integrated into the solid sample collection structure.

### CROSS-REFERENCE TO RELATED APPLICATIONS

This application claims the benefit of co-pending U.S. Provisional Patent Application Serial No. 63/698,119, filed September 24, 2024, which is hereby incorporated by reference in its entirety.

### FIELD

The present disclosure generally relates to sample collection devices and methods for using the same. Particularly, devices and methods described herein relate to multi-phase fecal collection.

### BACKGROUND

To perform testing on a biological sample, such as a fecal sample, a user may collect a sample for testing. Certain types of biological samples can often be multi-phase (i.e., the biological sample can include a liquid portion and a solid portion). Historically, tools for sample collection are designed to collect either a liquid portion of the sample or a solid portion of the sample. Collecting a solid portion of the sample for testing can involve engaging a sample collection device with a solid portion of the sample to collect and retain a solid portion of the sample. Tools such as a transfer pipette can be utilized to collect and retain a liquid portion of the sample. Because these sample collection tools can be limited to collection of a single phase of the sample, the sample volume collected and retained for testing may be limited. This can present challenges, as the sample collected must be a sufficient volume to perform the desired testing.

### SUMMARY

In an example, a device for collecting a multi-phase sample is disclosed. The device includes a solid sample capture device and a liquid sample capture device. The solid sample capture device includes a grip, an elongated member, and a solid sample collection structure. The liquid sample capture device is integrated into the solid sample capture device. The liquid sample capture device includes a suction component integrated into the grip, a tube integrated into the elongated member, and a tip integrated into the solid sample collection structure.

In another example, a method of collecting a multi-phase sample is disclosed. The method includes collecting a solid portion of a sample via a solid sample capture device. The solid sample capture device includes a grip, an elongated member, and a solid sample collection structure. The method further includes collecting a liquid portion of a sample via a liquid sample capture device. The liquid sample capture sample device is integrated into the solid sample capture device. The liquid sample capture device includes a suction component integrated into the grip, a tube integrated into the elongated member, and a tip integrated into the solid sample collection structure.

The features, functions, and advantages that have been discussed can be achieved independently in various examples or may be combined in yet other examples. Further details of the examples can be seen with reference to the following description and drawings.

### BRIEF DESCRIPTION OF THE DRAWINGS

Various aspects and features of the present disclosure are described hereinbelow with reference to the drawings wherein:

FIG. 1A illustrates a device for collecting a multi-phase sample, according to an example embodiment.

FIG. 1B illustrates a device for collecting a multi-phase sample, according to an example embodiment.

FIG. 1C illustrates a device for collecting a multi-phase sample, according to an example embodiment.

FIG. 1D illustrates a device for collecting a multi-phase sample, according to an example embodiment.

FIG. 2A illustrates a grip and a suction component of a device for collecting a multi-phase sample, according to an example embodiment.

FIG. 2B illustrates a grip and a suction component of a device for collecting a multi-phase sample, according to an example embodiment.

FIG. 3 illustrates a tip and solid sample capture structure of a device for collecting a multi-phase sample, according to an example embodiment.

FIG. 4 illustrates a method of collecting a multi-phase sample, according to an example embodiment.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

Within examples, the present disclosure is directed to devices and methods for collecting a sample, such as a multi-phase sample, for testing. Testing and/or analyzing, as referred to herein, may include, for example, capturing one or more images related to a biological sample. For example, testing can involve capturing images of a biological sample from an imaging sensor and determining a stain intensity. In another example, these images may come from competitive immunoassays for detection of antibodies in the biological sample and a competitive immunoassay.

Certain sample of types, such as fecal samples, are often multi-phase. Namely, the consistency of samples of fecal samples can vary greatly between samples (e.g., a dehydrated fecal sample, a fluid fecal sample, etc.). Historically, collection of a biological samples for testing involves a user (e.g., a clinician) collecting a single phase of a sample (e.g., solid or liquid). More particularly, tools for sample collection are designed to collect either a liquid portion of the sample or a solid portion of the sample. Collecting a solid portion biological sample for testing can involve engaging a sample collection device with a solid portion of the sample to collect and retain a solid portion of the sample. Alternatively, tools such as a transfer pipette can be utilized to collect and retain a liquid portion of the sample. These sample collection techniques can be limited to collection of a single phase of the sample, which can limit the sample volume collected and retained for testing. This can present challenges, as the sample collected must be a sufficient volume to perform the desired testing.

Moreover, handling certain types of crude samples, such as fecal samples, can be particularly problematic. Fecal samples, for instance, can contain harmful pathogens, including bacteria, viruses, and parasites, which can pose health risks to users handling them. As such, fecal samples can contaminate surfaces, equipment, and other samples if not handled properly. This contamination can compromise the accuracy of test results and pose risks to laboratory personnel and others who come into contact with the contaminated items.

The example devices and methods disclosed herein address some of these issues. An example device collects both solid and liquid portions of a sample. More particularly, a liquid sample capture device is integrated with a solid capture device to allow a user to collect both a liquid portion of the sample and a solid portion of the sample using a single device. The ability to collect both solid and liquid portions of the sample with a single device can increase the total volume of sample collected. Additionally, this can decrease the amount of time taken to collect an adequate sample volume.

Moreover, the example device described herein includes a separation between portions of the device that user interacts with (e.g., a grip and a suction component) from the portions of the device that engage with the sample (e.g., a solid sample capture structure and a tip). This helps prevent user contact with the sample and prevent contamination of the surrounding sample collection area. Further, this helps to prevent sample loss, sample contamination, and/or compromising of the sample.

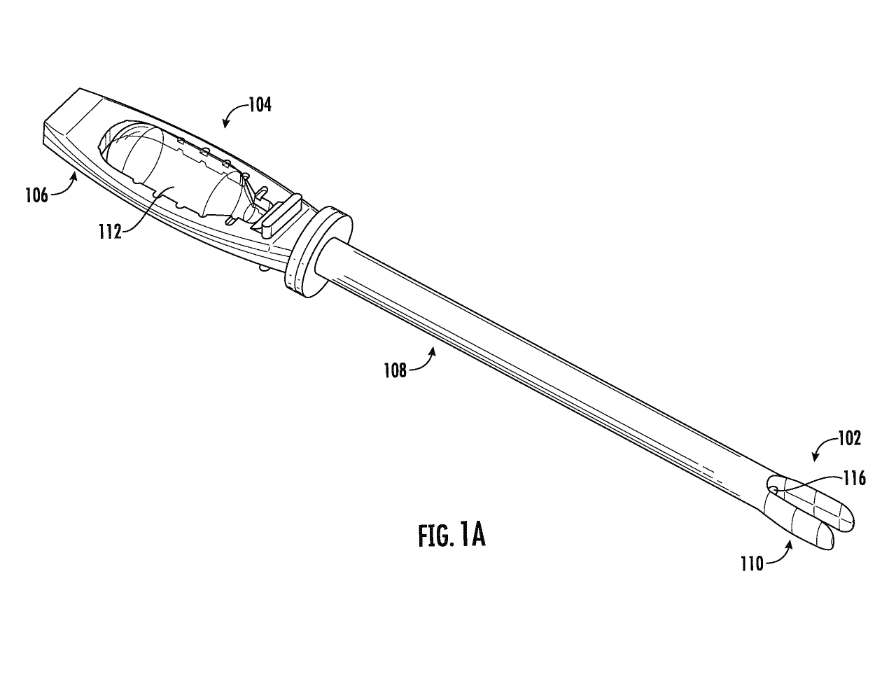

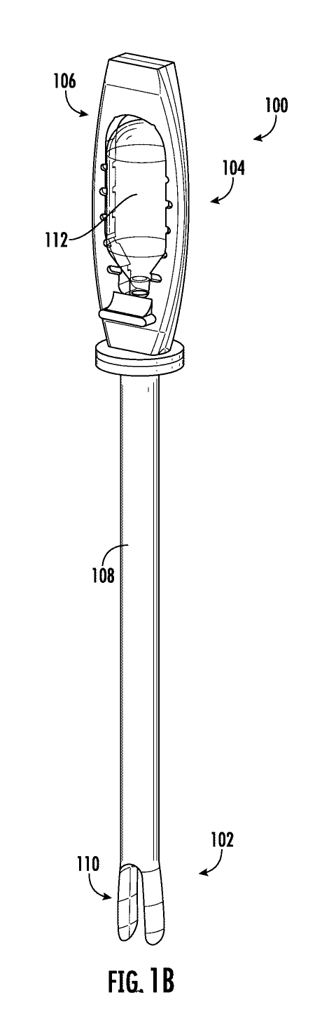

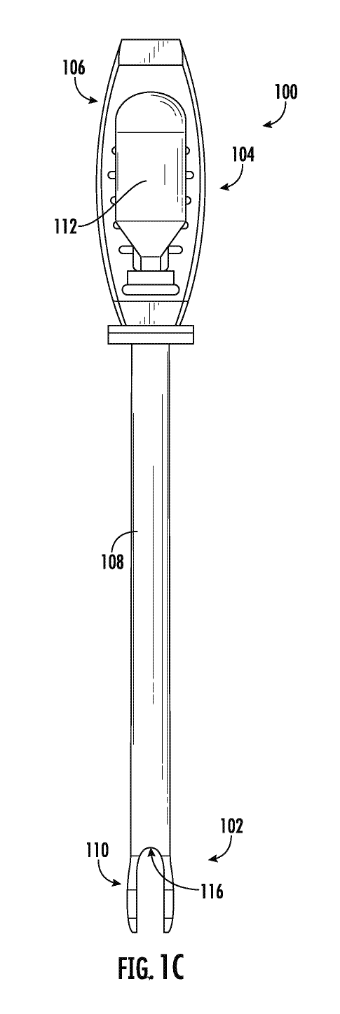

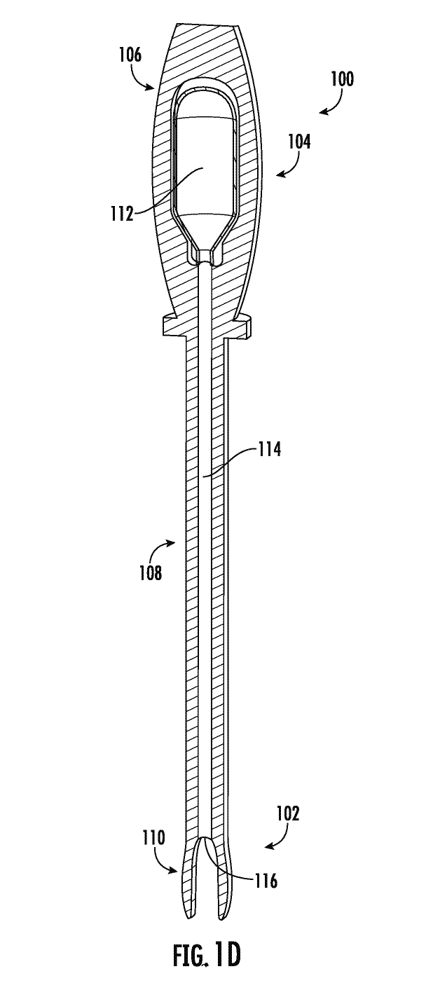

Now referring to the Figures, FIGS. 1A-1D illustrate device 100 for collecting a multi-phase sample, according to an example embodiment. An example device 100 includes a solid sample capture device 102 and liquid sample capture device 104. In examples, the liquid sample capture device 104 is integrated into the solid sample capture device 102.

In embodiments, the solid sample capture device 102 and the liquid sample capture device 104 are formed as a one-piece, monolithic structure. In other examples, the solid sample capture device 102 and the liquid sample capture device 104 can include multiple pieces coupled to and integrated with one another. Many example configurations are possible.

In example implementations, a solid sample capture device 102 can be utilized to collect a solid portion of the sample. A solid sample capture device 102 includes a grip 106 for user handling. The grip 106 is coupled to one end of an elongated member 108. The solid sample capture device 102 also includes a solid sample capture structure 110 coupled to the opposite end of the elongated member 108. The solid sample capture structure 110 can engage with the solid portion of the sample to collect and retain the sample.

In practice, a user handles the device 100 by way of the grip 106 (i.e., the user holds the device 100 using the grip 106). The grip 106 is spaced apart from the solid sample capture structure 110 by way of the elongated member 108. Namely, the grip 106 and the solid sample capture structure 110 are positioned at opposite ends of an elongated member 108. Positioning the grip 106 and solid sample capture structure 110 apart from one another prevents user contact and/or interaction with the sample. As noted above, samples, such as fecal samples, can contain harmful pathogens, including bacteria, viruses, and parasites, which can pose health risks to users handling them. Thus, preventing user contact and interaction with the sample is desirable.

In example implementations, the grip 106 is ergonomically shaped to facilitate a user handling the device 100. For instance, in some examples, the grip 106 can be curved. A curve in the grip 106 can increase a user’s handling ability and/or control of the device 100. This can also prevent slipping or dropping of the device 100 while handling. This can prevent sample loss, sample contamination, and/or compromising the sample, as well as prevent contamination and/or contact of the sample with the collection area and/or user.

The suction component 112 is integrated into the grip 106. In some examples, the grip 106 surrounds at least a portion the suction component 112. For instance, the suction component 112 may be a bulb (e.g., a pipette bulb) as shown in the examples in FIGS. 1A-1D. In these examples, the grip 106 may surround at least a portion of the surface of the suction component 112. Additionally or alternatively, the suction component 112 can include a hollow portion of the grip 106.

In examples, the grip 106 is coupled with a first end of the elongated member 108. The tube 114 is integrated into the elongated member 108. For instance, in some examples, the elongated member 108 surrounds at least a portion of the tube 114. Additionally or alternatively, the tube 114 can include a hollow portion of the elongated member 108.

The solid sample capture structure 110 is coupled to a second end of the elongated member 108, opposite the first end of the elongated member 108 coupled to the grip 106. In practice, the solid sample capture structure 110 engages with the sample to facilitate collecting and retaining a solid portion of a sample.

In example implementations, the solid sample capture structure 110 can include prongs or a fork. For instance, in the examples shown in FIG. 1A-1D, the solid sample capture structure 110 can include two prongs extending laterally away from the elongated member 108. Alternative examples may include any suitable number of prongs for collecting a solid portion of a sample. For example, the solid sample capture structure 110 may include one prong for collecting a solid portion of a sample. In other embodiments, the solid sample capture structure 110 may include three or more prongs. In embodiments, the prongs may be spaced apart from one another to define a space in which the solid portion of the sample is retained.

To collect a solid portion of a sample, the user can hold the device 100 using the grip 106. The user can then engage the solid sample capture structure 110 with the sample. The solid sample capture structure 110 can then collect and retain a solid portion of the sample. To dispense the solid portion of the sample, the user can engage the solid sample capture structure 110 with the desired device, for example, for testing and/or to prepare the sample for testing (e.g., a sample preparation device).

As noted above, device 100 further includes a liquid sample capture device 104 integrated into the solid sample capture device 102. In example implementations, the liquid sample capture device 104 includes a suction component 112, a tube 114, and a tip 116. The tip 116 is fluidly connected to the tube 114 and the tube 114 is fluidly connected to the suction component 112. In example configurations, one or more of these components are integrated into various components the solid sample capture device 102. For instance, the suction component 112 can be integrated into the grip 106. The tube 114 can be integrated into the elongated member 108. And the tip 116 can be integrated into the solid sample capture structure 110. In this manner, the device 100 can collect both solid and liquid portions of a sample in a single device.

As noted above, in example configurations, the suction component 112 is integrated into the grip 106. In some examples, the suction component 112 and the grip 106 are formed as a one-piece, monolithic structure. For instance, the suction component 112 may be a hollow portion of the grip 106. In other examples, the solid sample capture device 102 and the liquid sample capture device 104 can include multiple pieces coupled to and integrated with one another. For instance, the suction component 112 may be a bulb (e.g., a pipette bulb) as shown in the examples in FIGS. 1A-1D. In these examples, the grip 106 may surround at least a portion of the surface of the suction component 112. Many example configurations are possible.

The suction component 112 is configured to create a partial vacuum in the liquid sample capture device 102 when compressed. The suction component 112 can include compliant material to allow compression of the suction component 112. In some examples, the suction component 112 can include flexible plastics, rubber, or the like, such as low-density polyethylene (LDPE) or similar. Many example materials are possible.

The tube 114 is integrated into the elongated member 108 and fluidly connected to the suction component 112. In some examples, the tube 114 and the elongated member 108 are formed as a one-piece, monolithic structure. For instance, the tube 114 may be a hollow portion of the elongated member 108. In other examples, the tube 114 and the elongated member 108 can include multiple pieces coupled to and integrated with one another. For instance, the elongated member 108 may surround at least a portion of the surface of the tube 114. Many example configurations are possible.

The tip 116 is fluidly connected to the tube 114 and is integrated into the solid sample collection structure 110. The tip 116 engages with the sample so that a liquid portion of the sample can be drawn into the tube 114. In some example implementations, the tip 116 is a pipette tip, or similar. In examples, such as the examples shown in FIGS. 1A-1D, the tip 116 of the liquid capture device 104 can be positioned in between the prongs of the solid sample capture structure 110. In this manner, a user can collect and/or dispense both a solid portion of the sample and a liquid portion of the sample simultaneously. Many example configurations are possible.

To collect a liquid portion of a sample, a user can compress the suction component 112 to create a partial vacuum in the liquid sample capture device 104. The user can then release the suction component 112, drawing a liquid portion of the sample into the tube 114 via suction forces. In practice, the user can iteratively squeeze and release the suction component 112 until a desired volume is collected. Once a liquid portion of the sample is collected and/or a desired volume of liquid sample is collected, the user can release the suction component 112. The tube 114 can then retain the collected liquid portion of the sample via vacuum forces.

The tube 114 can retain the liquid portion of the sample from when it is collected until it is dispensed. In some examples, the tube 114 can retain a volume between 25-250 µL, for example. In some examples, this volume may be lower (e.g., 50 µL) and in some examples, this volume may be higher (e.g., 230 µL). Varying volumes may be more beneficial for a variety of reasons, such as different tests or different species. For instance, some tests may require a larger volume of sample. In these instances, a tube 114 with a greater volume may be desired. Alternatively, some tests may require a smaller volume of sample. In these examples, a tube 114 with a smaller volume may be desired.

In some examples, the tube 114 and/or the elongated member 108 can include measurement indicators of the volume of liquid. For instance, in an example, tube 114 and/or the elongated member 108 is clear or opaque so that the user can see the amount of sample collected. The tube 114 and/or elongated member 108 can include a scale indicating volume along the length of the tube 114 and/or adjacent to the tube 114. In this manner, the user can determine whether adequate volume of sample has been collected.

In some example implementations, the solid portion and liquid portion of the sample can be collected simultaneously. For instance, while the user compresses the suction component 112 to collect the liquid portion of the sample, the solid sample collection structure 110 may simultaneously be engaged with a solid portion of the sample. Thus, facilitating simultaneous collection of the solid portion and liquid portion of the sample. Additionally or alternatively, the user may collect a liquid portion of the sample first and then collect a solid portion of the sample, or vis versa.

In some example implementations, the collected sample may not be dispensed and/or used immediately. The device 100 includes materials that allow for transfer and storage of the collected sample.

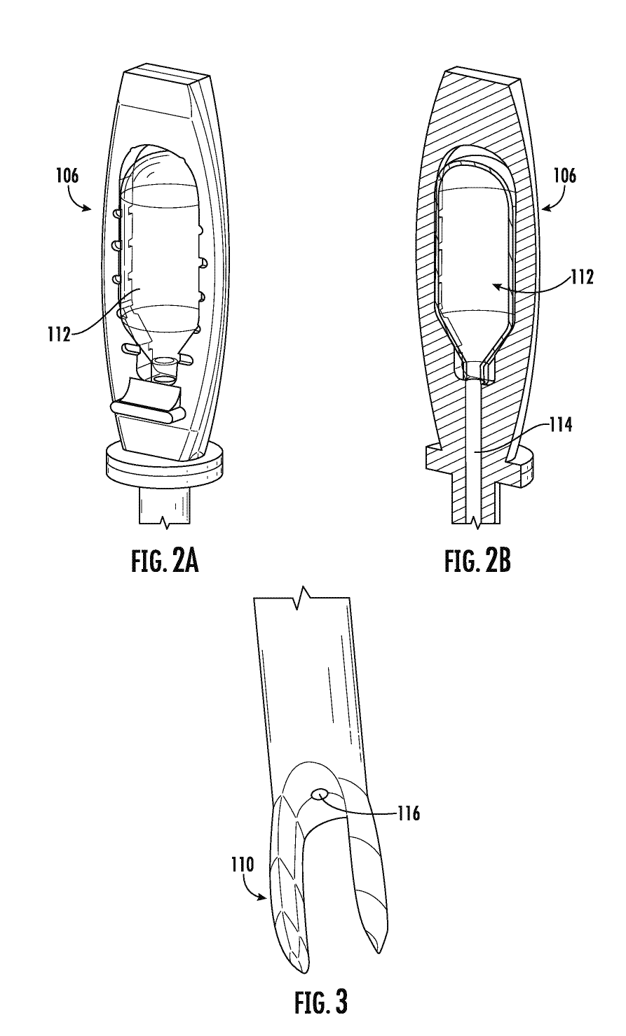

Now referring to FIGS. 2A-2B, which illustrate the grip 106 and the suction component 112 of the device 100. The suction component 112 is integrated into the grip 106. In some examples, the solid sample capture device 102 and the liquid sample capture device 104 can include multiple pieces coupled to and integrated with one another. For instance, the suction component 112 may be a bulb (e.g., a pipette bulb) as shown in the examples in FIGS. 2A-2B. In these examples, the grip 106 may surround at least a portion of the surface of the suction component 112. Alternatively, the suction component 112 and the grip 106 can be formed as a one-piece, monolithic structure. For instance, the suction component 112 may be a hollow portion of the grip 106. Many example configurations are possible.

Further, as shown in FIGS. 2A-2B, the suction component 112 is fluidly connected to the tube 114 so that compression of the suction component 112 can create a partial vacuum in the liquid capture device 104.

Now referring to FIG. 3, a solid sample capture structure 110 and a tip 116 of a device 100 for collecting a multi-phase sample, according to an example embodiment. In example implementations, the solid sample capture structure 110 can include prongs or a fork. In examples, such as the example shown in FIG. 3, the tip 116 of the liquid capture device 104 can be positioned in between the prongs of the solid sample capture structure 110. In this manner, a user can collect and/or dispense both a solid portion of the sample and a liquid portion of the sample simultaneously.

In some example embodiments, the sample can be used for a variety of tests. For instance, these tests may include imaging of one or more of the following: (i) fecal matter; (ii) blood; (iii) urine; (iv) saliva; (v) secretion; (vi) excretion; (vii) FNA; (viii) lavage fluids; (ix) body cavity fluids; (x) semen; (xi) ear wax; (xii) skin cells; (xiii) biopsied samples, (xiv) exotics; (xv) cultured cells; (xvi) bacteria; (xvii) worms; (xviii) parasites; and (xix) ear mites, among other possibilities. Test may additionally include one or more of the following: blood coagulation test, polymerase chain reaction (PCR) test, and/or immunoassay, among other possibilities. For example, in some example embodiments, these tests may include one or more of the following blood chemistry tests: SDMA, Total T4 (TT4), Bile Acids, C-reactive Protein (CRP), Progesterone, Fructosamine, and/or Phenobarbital (PHBR), among other possibilities. For example, in some example embodiments, these tests may include one or more of the following blood chemistry profile tests that measure one or more of the following: ALB, ALB/GLOB, ALKP, ALT, AMYL, AST, BUN, BUN/CREA, ca, CHOL, Cl<, Cl, CREA, CRP, FRU, GGT, GLOB, GLU, K, LAC, LDH, LIPA, Mg, Na, NH3, PHOS, TBIL, TP, TRIG and/or URIC, among other possibilities. Other examples are possible.

### EXAMPLE METHODS AND ASPECTS

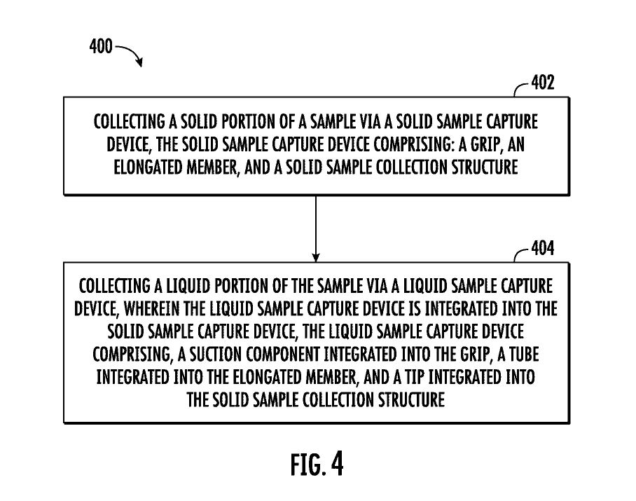

Now referring to FIG. 4, an example method of collecting a multi-phase sample is disclosed. Method 400 shown in FIG. 4 presents an example of a method for preparing a biological testing sample that could be used with the components shown in FIGS. 1A-3, for example. Further, devices or systems may be used or configured to perform logical functions presented in FIG. 4. In other examples, components of the devices and/or systems may be arranged to be adapted to, capable of, or suited for performing the functions, such as when operated in a specific manner. Method 400 may include one or more operations, functions, or actions as illustrated by one or more of blocks 402-404. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 402, method 400 involves collecting a solid portion of a sample via a solid sample capture device. The solid sample capture device includes a grip, an elongated member, and a solid sample collection structure.

In some examples, sample collection device comprises prongs or a fork.

At block 404, method 400 involves collecting a liquid portion of the sample via a liquid sample capture device. The liquid sample capture device is integrated into the solid sample capture device. The liquid sample capture device includes a suction component integrated into the grip, a tube integrated into the elongated member, and a tip integrated into the solid sample collection structure.

In some examples, the suction component comprises compliant material. In these examples, collecting the liquid portion of the sample comprises compressing the suction component.

In some examples, the suction component comprises compliant material. In these examples, dispensing the collected liquid portion of the sample, wherein dispensing the collected liquid portion of the sample comprises compressing the suction component.

In some examples, the sample is a feces sample.

In some examples, the liquid sample capture device is configured to capture between 25-250 µL of liquid sample.

In some examples, the liquid sample capture device retains liquid sample after the liquid sample is collected.

In some examples, the tip of the liquid sample capture device is fluidly connected to the tube, and wherein the tube is fluidly connected to the suction component.

The singular forms of the articles "a, an," and "the" include plural references unless the context clearly indicates otherwise. For example, the term "a compound" or "at least one compound" can include a plurality of compounds, including mixtures thereof.

Various aspects and embodiments have been disclosed herein, but other aspects and embodiments will be apparent to those skilled in the art. Additionally, the various aspects and embodiments disclosed herein are provided for explanatory purposes and are not intended to be limiting, with the true scope being indicated by the following claims.

1. A device for collecting a multi-phase sample, the device comprising:
a solid sample capture device comprising:
a grip,
an elongated member, and
a solid sample collection structure; and
a liquid sample capture device integrated into the solid sample capture device, the liquid sample capture device comprising,
a suction component integrated into the grip,
a tube integrated into the elongated member, and
a tip integrated into the solid sample collection structure.

2. The device of claim 1, wherein the multi-phase sample is a fecal sample.

3. The device of claim 1, wherein the liquid sample capture device is a pipette, wherein the suction component is a pipette bulb, and wherein the tip is a pipette tip.

4. The device of claim 1, wherein the suction component comprises compliant material.

5. The device of claim 1, wherein the suction component is hollow portion of the grip.

6. The device of claim 1, wherein the solid sample collection structure comprises prongs or a fork.

7. The device of claim 1, wherein the liquid sample capture device is configured to capture between 25-250 µL of liquid sample.

8. The device of claim 1, wherein the liquid sample capture device retains a collected liquid portion of a sample.

9. The device of claim 1, wherein the tip of the liquid sample capture device is fluidly connected to the tube, and wherein the tube is fluidly connected to the suction component.

10. The device of claim 1, wherein the tip of the liquid sample capture device is adjacent to the solid sample collection structure.

11. The device of claim 1, wherein at least one of the tube or the elongated member comprise measurement indicators of a volume of liquid sample.

12. A method of collecting a multi-phase sample using a sample collection device, the method comprising:
collecting a solid portion of a sample via a solid sample capture device, the solid sample capture device comprising:
a grip,
an elongated member, and
a solid sample collection structure; and
collecting a liquid portion of the sample via a liquid sample capture device, wherein the liquid sample capture device is integrated into the solid sample capture device, the liquid sample capture device comprising,
a suction component integrated into the grip,
a tube integrated into the elongated member, and
a tip integrated into the solid sample collection structure.

13. The method of claim 12, wherein the suction component comprises compliant material, and wherein collecting the liquid portion of the sample comprises compressing the suction component.

14. The method of claim 12, wherein the suction component comprises compliant material, and wherein the method further comprises:
dispensing the collected liquid portion of the sample, wherein dispensing the collected liquid portion of the sample comprises compressing the suction component.

15. The method of claim 12, wherein the sample is a fecal sample.

16. The method of claim 12, wherein the liquid sample capture device is a pipette, wherein the suction component is a pipette bulb, and wherein the tip is a pipette tip.

17. The method of claim 12, wherein the sample collection device comprises prongs or a fork.

18. The method of claim 12, wherein the liquid sample capture device is configured to retain between 25-250 µL of liquid sample.

19. The method of claim 12, wherein the tip of the liquid sample capture device is fluidly connected to the tube, and wherein the tube is fluidly connected to the suction component.

20. The method of claim 12, wherein the tip of the liquid sample capture device is adjacent to the solid sample collection structure.
